(19) United States
(12) Patent Application Publication
(10) Pub. No.: US 2017/0196957 A1
(43) Pub. Date: Jul. 13, 2017

(54) RECOMBINANT T CELL RECEPTOR LIGAND COMPOSITIONS AND METHODS FOR TREATMENT OF PROSTATE CANCER

(71) Applicants: Oregon Health & Science University, Portland, OR (US); The United States Government as Represented by the Department of Veterans Affairs, Washington, DC (US); University of Maryland, Baltimore, Baltimore, MD (US)

(72) Inventors: Arthur A. Vandenbark, Portland, OR (US); Roberto Meza-Romero, Beaverton, OR (US); Richard Alexander, Ellicott City, MD (US); Elena Klyushnenkova, Baltimore, MD (US)

(73) Assignees: Oregon Health & Science University, Portland, OR (US); The United States Government as Represented by the Department of Veterans Affairs, Washington, DC (US); University of Maryland, Baltimore, Baltimore, MD (US)

(21) Appl. No.: 15/400,587

(22) Filed: Jan. 6, 2017

Related U.S. Application Data
(60) Provisional application No. 62/276,709, filed on Jan. 8, 2016.

(51) Int. Cl. A61K 39/00; C12N 9/64; C07K 14/74

(52) U.S. Cl. CPC A61K 39/0011; C07K 14/70539; C12N 9/6445; C12Y 304/21077; A61K 2039/605; A61K 2039/627; C07K 2319/40

(57) ABSTRACT

Disclosed herein are compositions and methods for treating or inhibiting prostate cancer. The compositions include a MHC molecule including covalently linked first and second domains, wherein the first domain is an MHC class II β1 domain and the second domain is an MHC class II α1 domain, wherein the amino terminus of the α1 domain is covalently linked to the carboxy terminus of the β1 domain, and a prostate specific antigen peptide covalently linked to the first domain. The methods include administering a disclosed MHC molecule to a subject with prostate cancer.

### CROSS REFERENCE TO RELATED APPLICATION

This claims the benefit of U.S. Provisional Application No. 62/276,709, filed Jan. 8, 2016, which is incorporated herein by reference in its entirety.

### FIELD

This disclosure relates to cancer, and particularly to compositions and methods for treating prostate cancer, for example utilizing recombinant T cell receptor ligands.

### BACKGROUND

Prostate cancer is the most common cancer of men in the United States. There are no established therapies for the disease when the cancer has extended outside of the prostate gland. Such patients are at the highest risk of death from prostate cancer and limited treatment options exist for these men. As opposed to most other cancers, men with prostate cancer outside of the gland may have a long interval of overall wellness and remain immunologically intact. The goal of immunotherapy for these men is therefore to keep them in such a state despite their cancer and to shift the life expectancy of the treated population to that of the general population.

The immunotherapy of cancer is based on the expectation that the immune system can be manipulated to attack and destroy tumors. Many attempts to stimulate an immune response in order to do this have been attempted; very few have reached clinical use. It is increasingly clear that an immunosuppressive process is associated with the tumor-bearing state; therefore, attempts to stimulate an immune response have been subverted by this pre-existing immunosuppression. This is a major impediment to progress in the field of cancer immunotherapy.

Since tumors are derived from self, it appears that part of the immune suppression associated with tumors is due to the peripheral tolerance of self antigens. One of the critical cell populations responsible for this self-tolerance is the regulatory CD4 T lymphocyte, or T_{reg}. A direct role for T_{reg} function in cancer has been shown in multiple animal models and in humans by using functional blockade or depletion (reviewed in Savage et al., Trends Immunol. 34:33-40, 2013). Exposure of the immune system to growing tumors preferentially stimulates CD4⁺ regulatory T cells (T_{reg}) that normally prevent expansion of protective cytotoxic CD8⁺ T effector cells (T_{eff}). Approaches that can neutralize tumor-specific T_{reg} cells could allow emergence of the cytotoxic T_{eff} cells that reject the tumor in vivo. However, modulating T_{reg} function has not been particularly successful in human studies; all existing strategies target polyclonal T_{reg}, which have led to severe autoimmune reactions.

### SUMMARY

Disclosed herein are compositions including recombinant nucleic acid and polypeptide molecules including a major histocompatibility complex (MHC) molecule (such as a MHC class II β1α1 construct or a MHC class II α1 construct) and a tumor antigen. In some examples, the compositions include an MHC class II β1 domain covalently linked to an MHC class II α1 domain, wherein the carboxy terminus of the β1 domain is linked to the amino terminus of the α1 domain, and a tumor antigen peptide covalently linked to the β1 domain (for example, at or near the amino terminus of the β1 domain). In particular embodiments, the compositions include a DR2 recombinant T cell receptor ligand (RTL) including a prostate specific antigen (PSA) peptide (referred to herein as DR2/PSA-RTLs). In some examples, the disclosed constructs include the α1 and β1 domains of an HLA-DR2 molecule linked covalently to a PSA peptide. In some embodiments, the PSA peptide is a peptide that can induce PSA tumor-specific CD4⁺ T_{regs}. Exemplary PSA peptides utilized in the DR2/PSA-RTL constructs include PSA residues 221-240 or 171-190, or variants thereof (e.g., SEQ ID NOs: 1-3).

Also disclosed herein are methods of treating or inhibiting cancer (e.g., prostate cancer) in a subject using the MHC molecule-tumor antigen constructs. In some embodiments, the methods include administering an effective amount of a composition including a disclosed RTL construct (such as any one of SEQ ID NOs: 6-10) to a subject with prostate cancer. In some examples, the RTL construct blocks the function of PSA tumor-specific T_{regs} and/or increases cytotoxic PSA-specific CD8⁺ T_{eff} cells, which promotes tumor cell killing and/or tumor rejection.

The foregoing and other features of the disclosure will become more apparent from the following detailed description, which proceeds with reference to the accompanying figures.

### BRIEF DESCRIPTION OF THE DRAWINGS

FIGS. 1A and 1B are graphs showing response of DR2b×PSA F1 mice to TRAMP-PSA tumor cells. For both FIGS. 1A and 1B, DR2b×PSA F1 mice were injected intravenously with depleting anti-CD25 mAb (clone PC61) three days prior to inoculation with TRAMP-PSA tumor cells. FIG. 1A is a bar graph showing the CD8 T cell response to PSA, evaluated two weeks after tumor inoculation by IFN-γ ELISPOT. Bars show mean and standard deviation of triplicates (three mice per group pooled). ***** p=0.00001 by a 2-sided t-test compared to corresponding samples in a no treatment group. FIG. 1B is a “time to event” plot of tumor growth. Tumor growth was measured weekly and plotted according to a “time to event” analysis, where the event was a tumor base area of 100 mm². The p value for CD25 mAb-treated animals versus rat IgG negative control is shown.

FIG. 2 is a bar graph of the results where DR2b×B6-PSA F1 male mice (8-12 weeks old) were inoculated subcutaneously (s.c.) in the dorsal neck area with TRAMP-PSA tumor cells (3×10⁶ cells per mouse). DR2b/PSA₂₂₁₋₂₃₆ RTL (100 μg/dose in 100 μl) or vehicle were injected s.c. in the dorsal neck area on days −5, −4, −3, −2, −1, +8, +9, +11. Draining lymph nodes (cervical and axillary) were harvested two weeks after tumor inoculation, and lymphocytes were plated and cultured with peptide PSA₆₅₋₇₃ (HCIRNKSVI; SEQ ID NO: 11) (described in Pavlenko et al., Prostate 64:50-59, 2005) or irrelevant peptide Neo₄₉₋₅₉ (SSPVNSLRNVV; SEQ ID NO: 12), irradiated TRAMP-PSA (irTRAMP-PSAg) or control TRAMP-C1g (WT) tumor cells. ELISPOT assay for IFNγ was performed. Data are mean±SD of triplicate determinations.

FIG. 3 is a digital image of a Coomassie stained gel depicting the last step of PSA-RTL purification.

### SEQUENCE LISTING

Any nucleic acid and amino acid sequences listed herein or in the accompanying sequence listing are shown using standard letter abbreviations for nucleotide bases and amino acids, as defined in 37 C.F.R. §1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Jan. 5, 2017, and is 13,860 bytes, which is incorporated by reference herein.

SEQ ID NO: 1 is the amino acid sequence of PSA₂₂₁₋₂₄₀ 67C antigen.

SEQ ID NO: 2 is the amino acid sequence of PSA₂₂₁₋₂₄₀ 67S antigen.

SEQ ID NO: 3 is the amino acid sequence of PSA₁₇₁₋₁₉₀.

SEQ ID NO: 4 is the amino acid sequence of a DR2-5D (β1α1) construct.

SEQ ID NO: 5 is the amino acid sequence of an exemplary peptide linker.

SEQ ID NO: 6 is the amino acid sequence of a construct comprising PSA₂₂₁₋₂₄₀ 67C antigen, a linker, and DR2-5D.

SEQ ID NO: 7 is an exemplary nucleic acid sequence that encodes the construct of SEQ ID NO: 6.

SEQ ID NO: 8 is the amino acid sequence of a construct comprising PSA₂₂₁₋₂₄₀ 67S antigen, a linker and DR2-5D.

SEQ ID NO: 9 is an exemplary nucleic acid sequence that encodes the construct of SEQ ID NO: 8.

SEQ ID NO: 10 is the amino acid sequence of a construct comprising PSA171-190 antigen, a linker, and DR2-5D.

SEQ ID NO: 11 is the amino acid sequence of PSA₆₅₋₇₃.

SEQ ID NO: 12 is the amino acid sequence of Neo₄₉₋₅₉.

SEQ ID NO: 13 is the amino acid sequence of an exemplary DR2 β1α1 construct.

SEQ ID NOs: 14-17 are amino acid sequences of additional exemplary PSA peptides.

### DETAILED DESCRIPTION

The immunotherapy of cancer is based on the expectation that the immune system can be manipulated to attack and destroy tumors. Many attempts to stimulate an immune response in order to do this have been attempted; very few have reached clinical use. It is increasingly clear that an immunosuppressive process is associated with the tumor-bearing state; therefore, attempts to stimulate an immune response appear to have been subverted by this pre-existing immunosuppression. This is a major impediment to progress in the field of cancer immunotherapy.

Previous mouse studies utilizing Transgenic Adenocarcinoma of Mouse Prostate (TRAMP) tumors expressing the human tumor antigen PSA (TRAMP-PSA) in transgenic Human Leukocyte Antigen (HLA)-DR2b mice provided some insight (Klyushnenkova et al., J. Immunol. 182:1242-1246, 2009). In this study, when a CD4 T cell response to the tumor antigen was possible, the response was suppression of the cytotoxic T cell (CTL) response against the tumor antigen and the tumor grew rapidly. If a CD4 T cell response was not possible then no suppression was seen, a vigorous CTL response to the tumor antigen was found, and the tumor was rejected. Thus, the principal CD4 T cell response to the tumor antigen was suppression, consistent with peripheral tolerance (Klyushnenkova et al., J. Immunol. 182:1242-1246, 2009).

There are many cases where effector CD4 T cells represent the principal CD4 T cell response to an antigen, such as the autoimmune disease Multiple Sclerosis (MS). Effector CD4 T cells directed at peptides derived from nerve sheath proteins are a primary mediator of damage to the nervous system in MS. Methods to block these CD4 T cells lead to improvement of the disease. This is very different from that observed in the cancer model discussed above, where the primary CD4 T cell response was suppressive, probably because the tumor is viewed as self. Hence, as described herein, blocking the primarily suppressive CD4 T cell response to cancer antigens can be exploited as a therapy.

Disclosed herein are compositions that block specific CD4 T cells recognizing a tumor antigen. Since the primary CD4 response is regulatory, blocking of such CD4 T cells can result in removal of suppression and release of an effector CTL response to reject the tumor. As disclosed herein, this can be done by using a recently developed technology called Recombinant T cell Receptor Ligands (RTL) or partial MHC constructs including an MHC class II α1 polypeptide. RTLs comprise an α1 domain and a β1 domain of an MHC class II molecule linked into a single polypeptide chain with covalently coupled peptides attached by a linker or disulfide bond(s). RTLs containing neuronal protein antigens have been extensively evaluated in MS and clinical trials for this indication are underway (Yadav et al., Autoimmune Dis. 2012:954739, 2012). Treatment with RTLs in this case blocks the primarily effector CD4 T cell response to specific neuronal protein antigens and reverses autoimmune disease (Huan et al., J. Immunol. 172:4556-4566, 2004).

### I. Terms

Unless otherwise explained, all technical and scientific terms used herein have the same meaning as commonly understood by one of ordinary skill in the art to which this disclosure belongs. Definitions of common terms in molecular biology may be found in Krebs et al., Lewin's Genes XI, published by Jones and Bartlett Learning, 2012 (ISBN 1449659853); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 2011 (ISBN 8126531789); and George P. Rédei, Encyclopedic Dictionary of Genetics, Genomics, and Proteomics, 2nd Edition, 2003 (ISBN: 0-471-26821-6).

The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 8 amino acids (such as about 8-50 or 8-23 amino acids) in a unique spatial conformation. Methods of determining spatial conformation of epitopes include, for example, x-ray crystallography and two-dimensional nuclear magnetic resonance.

An antigen can be a tissue-specific antigen or a disease-specific antigen. These terms are not exclusive, as a tissue-specific antigen can also be a disease-specific antigen. A tissue-specific antigen is expressed in a limited number of tissues, such as a single tissue or may be expressed by more than one tissue. One example of a tissue-specific antigen is an antigen of the prostate (such as PSA). A disease-specific antigen is expressed coincidentally with a disease process. Specific non-limiting examples of disease-specific antigens are an antigen whose expression correlates with, or is predictive of, prostate cancer. One exemplary antigen is prostate specific antigen (PSA), or a portion thereof.

Domain: A discrete part of an amino acid sequence of a polypeptide or protein that can be equated with a particular function. For example, the α and β polypeptides that constitute a MHC class II molecule are each recognized as having two domains, α1, α2 and β1, β2, respectively. The various domains in each of these molecules are typically joined by linking amino acid sequences. In one embodiment, the entire domain sequence is included in a recombinant molecule by extending the sequence to include all or part of the linker or the adjacent domain. For example, when selecting the α1 domain of an MHC class II molecule, the selected sequence may extend from amino acid residue number 1 of the α chain, through the entire α1 domain and include all or part of the linker sequence located at about amino acid residues 76-90 (at the carboxy terminus of the α1 domain, between the α1 and α2 domains). The precise number of amino acids in the various MHC molecule domains varies depending on the species of mammal, as well as between classes of genes within a species. The critical aspect for selection of a sequence for use in a recombinant molecule is the maintenance of the domain function rather than a precise structural definition based on the number of amino acids. One of skill in the art will appreciate that domain function may be maintained even if somewhat less than the entire amino acid sequence of the selected domain is utilized. For example, a number of amino acids at either the amino or carboxy termini of the α1 domain may be omitted without affecting domain function. Typically however, the number of amino acids omitted from either terminus of the domain sequence will be no greater than 10, and more typically no greater than 5 amino acids.

The functional activity of a particular selected domain may be assessed in the context of the two-domain MHC polypeptides provided by this disclosure (e.g., the MHC class II β1α1 polypeptides) using the antigen-specific T-cell proliferation assay as described below. For example, to test a particular β1 domain, the domain will be linked to a functional α1 domain so as to produce a β1α1 molecule and then tested in the described assay. A biologically active β1α1 polypeptide will inhibit antigen-specific T-cell proliferation by at least about 50%, thus indicating that the component domains are functional. Typically, such polypeptides will inhibit T-cell proliferation in this assay system by at least 75% and sometimes by greater than about 90%.

Isolated: An “isolated” biological component (such as a nucleic acid, peptide, or protein) has been substantially separated, produced apart from, or purified away from other biological components, e.g., other chromosomal and extrachromosomal DNA and RNA, and proteins. Nucleic acids, peptides, and proteins which have been “isolated” thus include nucleic acids and proteins purified by standard purification methods. The term also embraces nucleic acids, peptides, and proteins prepared by recombinant expression in a host cell, as well as chemically synthesized peptides, proteins, or nucleic acids.

Linker: A chemical structure or amino acid sequence that covalently links two polypeptide domains. Linker amino acid sequences may be included in the recombinant MHC polypeptides of the present disclosure to provide rotational freedom to the linked polypeptide domains and thereby to promote proper domain folding and inter- and intra-domain bonding. By way of example, in a recombinant polypeptide comprising Ag-β1-α1 (where Ag=antigen), linker sequences may be provided between the Ag and β1 domains and/or between β1 and α1 domains. Recombinant linker sequences, which are generally between 2 and 25 amino acids in length, are well known in the art and include, but are not limited to, the glycine(4)-serine spacer described by Chaudhary et al. (Nature 339:394-397, 1989).

MHC Class II: MHC class II molecules are formed from two non-covalently associated proteins, the α chain and the β chain. The α chain comprises α1 and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the antigen presenting cell (APC). MHC class II complexes, when associated with antigen (and in the presence of appropriate co-stimulatory signals) stimulate CD4 T-cells. The primary functions of CD4 T-cells are to initiate the inflammatory response, to regulate other cells in the immune system, and to provide help to B cells for antibody synthesis.

In some examples disclosed herein, an MHC class II β1α1 polypeptide includes a recombinant polypeptide comprising the α1 and β1 domains of a MHC class II molecule in covalent linkage. In other examples, a β1α1 nucleic acid includes a recombinant nucleic acid sequence encoding a β1α1 polypeptide. To ensure appropriate conformation, the orientation of the polypeptide is such that the carboxy terminus of the β1 domain is covalently linked to the amino terminus of the α1 domain. In one embodiment, the polypeptide is a human β1α1 polypeptide, and includes the α1 and β1 domains for a human MHC class II molecule. One specific, non-limiting example of a human β1α1 polypeptide is a molecule wherein the carboxy terminus of the β1 domain is covalently linked to the amino terminus of the α1 domain of an HLA-DR molecule. In one embodiment, the β1α1 polypeptide does not include a β2 domain. In another embodiment, the β1α1 polypeptide does not include an α2 domain. In yet another embodiment, the β1α1 polypeptide does not include either an α2 or a β2 domain.

Pharmaceutically acceptable carriers: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, and the like, for example sodium acetate or sorbitan monolaurate.

Prostate cancer: A malignant tumor, generally of glandular origin, of the prostate. Prostate cancers include adenocarcinomas and small cell carcinomas. Many prostate cancers express prostate specific antigen (PSA).

Prostate cancer initially grows in an androgen-dependent manner, and androgen deprivation therapy (ADT) is an effective treatment in many cases of prostate cancer. However, prostate cancer can eventually become refractory to ADT. “Castration-resistant prostate cancer” (CRPC, also known as hormone-refractory prostate cancer) is prostate cancer that has become androgen-independent and progresses despite low levels of androgens (for example, in a subject undergoing ADT).

Prostate specific antigen (PSA): Also known as kallikrein related peptidase 3 (KLK3). A serine protease present in seminal plasma that is thought to function in the liquefaction of seminal coagulum. PSA levels in serum are utilized clinically as a marker for diagnosis and/or monitoring of prostate cancer.

PSA nucleic acid and amino acid sequences are publicly available. Exemplary human PSA nucleic acid sequences include GenBank Accession Nos. NM_001030047, NM_001648, NM_001030048 and exemplary human PSA amino acid sequences include GenBank Accession Nos. NP_001025218, NP_001639, and NP_001025219, all of which are incorporated by reference herein as present in GenBank on Jan. 8, 2016.

Purified: The term purified does not require absolute purity; rather, it is intended as a relative term. Thus, for example, a purified peptide or protein preparation is one in which the peptide or protein is more enriched than the peptide or protein is in its natural environment within a cell or in an initial preparation. Preferably, a preparation is purified such that the protein or peptide represents at least 50% of the total peptide or protein content of the preparation. In some embodiments, a purified preparation contains at least 60%, at least 70%, at least 80%, at least 85%, at least 90%, at least 95% or more of the protein or peptide.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Subject: Living multi-cellular vertebrate organisms, a category that includes both human and non-human mammals.

T Cell: A white blood cell critical to the immune response. T cells include, but are not limited to, CD4⁺ T cells and CD8⁺ T cells. A CD4⁺ T lymphocyte is an immune cell that carries a marker on its surface known as cluster of differentiation 4 (CD4). These cells, classically known as helper T cells (Th cells), help orchestrate the immune response, including antibody responses as well as killer T cell responses. CD8⁺ T cells carry the cluster of differentiation 8 (CD8) marker. In one embodiment, CD8⁺ T cells are cytotoxic T lymphocytes (CTLs) which are capable of lysing target cells by direct cell contact. These cells play a role in the elimination of virus-infected cells and tumor cells, and are involved in transplant rejection processes. In another embodiment, a CD8 cell is a suppressor T cell. Mature T cells express CD3. Regulatory T cells (T_{reg}) suppress immune responses of other cells. In one example, a regulatory T cell is CD4⁺CD25⁺ that suppresses an immune response. In additional examples, a regulatory T cell expresses CD4, CD25, and FOXP3. In some examples, effector T cells (T_{eff}) include antigen-specific CTLs.

Treating or inhibiting: “Treating” a condition refers to a therapeutic intervention that ameliorates a sign or symptom of a disease or pathological condition, for example, prostate cancer, after it has begun to develop. “Inhibiting” refers to inhibiting the full development of the disease or condition. Inhibition of a condition can span the spectrum from partial inhibition to substantially complete inhibition (e.g., including, but not limited to prevention) of the condition. In some examples, the term “inhibiting” refers to reducing or delaying the onset or progression of a disease. A subject to be administered a therapeutically effective amount of the disclosed compositions can be identified by standard diagnosing techniques for such a disorder, for example, based on signs and symptoms, family history, and/or risk factors to develop the disease or disorder.

### II. MHC Protein-Antigen Constructs

The disclosed methods utilize MHC molecules (such as an RTL) linked to a tumor antigen in methods of treatment of cancer. Although the disclosure utilizes MHC class II β1α1 polypeptides to exemplify the compositions, additional MHC constructs, such as an MHC class II α1 polypeptide (e.g., as described in U.S. Pat. App. Publ. No. 2015/0044245, incorporated herein by reference in its entirety), are also contemplated for use in the disclosed compositions and methods.

RTLs are monomeric recombinant polypeptides that can mimic MHC function and include only those MHC domains that define an antigen binding cleft. The RTLs are capable of antigen-specific T-cell binding and include, in the case of human class II MHC molecules, only the α1 and β1 domains in covalent linkage (and in some examples in association with an antigenic determinant). For convenience, such MHC class II RTL polypeptides are hereinafter referred to as “β1α1” polypeptides. These two domain molecules may be readily produced by recombinant expression in prokaryotic or eukaryotic cells, and readily purified in large quantities. Moreover, these molecules may easily be loaded with any desired peptide antigen.

A. Recombinant MHC Class II Dial Molecules

The amino acid sequences of mammalian MHC class II α and β chain proteins, as well as nucleic acids encoding these proteins, are well known in the art and available from numerous sources including GenBank. Exemplary sequences are provided in Auffray et al. (Nature 308:327-333, 1984) (human HLA DQ α); Larhammar et al. (Proc. Natl. Acad. Sci. USA 80:7313-7317, 1983) (human HLA DQ β); Das et al. (Proc. Natl. Acad. Sci. USA 80:3543-3547, 1983) (human HLA DR α); Tonnelle et al. (EMBO J. 4:2839-2847, 1985) (human HLA DR β); Lawrance et al. (Nucl. Acids Res. 13:7515-7528, 1985) (human HLA DP α); Kelly and Trowsdale (Nucl. Acids Res. 13:1607-1621, 1985) (human HLA DP β); Syha et al. (Nucl. Acids Res. 17:3985, 1989) (rat RT1.B α); Syha-Jedelhauser et al. (Biochim. Biophys. Acta 1089:414-416, 1991) (rat RT1.B β); Benoist et al. (Proc. Natl. Acad. Sci. USA 80:534-538, 1983) (mouse I-A α); Estess et al. (Proc. Natl. Acad. Sci. USA 83:3594-3598, 1986) (mouse I-A β), all of which are incorporated by reference herein. In a particular embodiment, the MHC class II protein is a human HLA-DR, such as HLA-DR2b (also referred to as HLA-DRB1*1501; e.g., GenBank Accession Nos. NM_002124 and NP_002115, incorporated herein by reference as present in GenBank on Jan. 8, 2016).

The recombinant MHC class II molecules of the present disclosure include the β1 domain of the MHC class II β chain covalently linked to the α1 domain of the MHC class II α chain. The α1 and β1 domains are well defined in mammalian MHC class II proteins. In some examples, MHC class II α chains include a leader sequence that is involved in trafficking the polypeptide and is proteolytic ally removed to produce the mature α polypeptide. Typically, the α1 domain is regarded as comprising about residues 1-90 of the mature chain. The native peptide linker region between the α1 and α2 domains of the MHC class II protein spans from about amino acid 76 to about amino acid 93 of the mature α chain, depending on the particular α chain under consideration. Thus, an α1 domain may include about amino acid residues 1-90 of the mature α chain, but one of skill in the art will recognize that the C-terminal cut-off of this domain is not necessarily precisely defined, and, for example, might occur at any point between amino acid residues 70-100 of the α chain. In some examples, the α1 domain includes amino acids 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of a mature MHC class II α domain. In other examples, an al domain includes about residues 20-120 (such as about residues 20-110, 24-110, 24-109, 25-100, 25-109, 26-110, 26-109, 30-120, 32-120, 32-115, 26-90, 26-85, 26-84, or other overlapping regions) of the full length MHC class II α polypeptide. In some examples, the MHC class II α1 domain does not include an N-terminal methionine; however, an N-terminal methionine can be present, for example as a result of expression in a bacterial, yeast, or mammalian system, or the N-terminal methionine may subsequently be removed. The composition of the α1 domain may also vary outside of these parameters depending on the mammalian species and the particular α chain in question. One of skill in the art will appreciate that the precise numerical parameters of the amino acid sequence are less important than the maintenance of domain function.

Similarly, the β1 domain is typically regarded as comprising about residues 1-90 of the mature β chain. The linker region between the β1 and β2 domains of the MHC class II protein spans from about amino acid 85 to about amino acid 100 of the β chain, depending on the particular β chain under consideration. Thus, the (31 protein may include about amino acid residues 1-100, but one of skill in the art will again recognize that the C-terminal cut-off of this domain is not necessarily precisely defined, and, for example, might occur at any point between amino acid residues 75-105 of the β chain. In some examples, the β1 domain includes amino acids 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of a mature MHC class II β chain. In some examples, the MHC class II β1 domain does not include an N-terminal methionine; however, an N-terminal methionine can be present, for example as a result of expression in a bacterial, yeast, or mammalian system. The composition of the β1 domain may also vary outside of these parameters depending on the mammalian species and the particular β chain in question. Again, one of skill in the art will appreciate that the precise numerical parameters of the amino acid sequence are less important than the maintenance of domain function.

In one embodiment, the β1α1 molecules do not include a β2 domain. In another embodiment, the β1α1 molecules do not include an α2 domain. In yet a further embodiment, the β1α1 molecules do not include either an α2 or a β2 domain. In some examples, the MHC class II β1α1 polypeptide does not include an N-terminal methionine; however, an N-terminal methionine can be present, for example as a result of expression in a bacterial, yeast, or mammalian system, or the N-terminal methionine may subsequently be removed.

Nucleic acid molecules encoding these domains may be produced by standard means, such as amplification by the polymerase chain reaction (PCR). Standard approaches for designing primers for amplifying open reading frames encoding these domains may be employed. Libraries suitable for the amplification of these domains include, for example, cDNA libraries prepared from the mammalian species in question; such libraries are available commercially, or may be prepared by standard methods. Thus, for example, constructs encoding the β1 and α1 polypeptides may be produced by PCR using four primers: primers B1 and B2 corresponding to the 5′ and 3′ ends of the 31 coding region, and primers A1 and A2 corresponding to the 5′ and 3′ ends of the al coding region. Following PCR amplification of the (31 and α1 domain coding regions, these amplified nucleic acid molecules may each be cloned into standard cloning vectors, or the molecules may be ligated together and then cloned into a suitable vector. To facilitate convenient cloning of the two coding regions, restriction endonuclease recognition sites may be designed into the PCR primers. For example, primers B2 and A1 may each include a suitable site such that the amplified fragments may be readily ligated together following amplification and digestion with the selected restriction enzyme. In addition, primers B1 and A2 may each include restriction sites to facilitate cloning into the polylinker site of the selected vector. Ligation of the two domain coding regions is performed such that the coding regions are operably linked, e.g., to maintain the open reading frame. Where the amplified coding regions are separately cloned, the fragments may be subsequently released from the cloning vector and gel purified, preparatory to ligation.

In certain embodiments, a peptide linker is provided between the β1 and α1 domains. Typically, this linker is between 2 and 25 amino acids in length, and serves to provide flexibility between the domains such that each domain is free to fold into its native conformation. The linker sequence may conveniently be provided by designing the PCR primers to encode the linker sequence. Thus, in the example described above, the linker sequence may be encoded by one of the B2 or A1 primers, or a combination of each of these primers.

An exemplary β1α1 DR2 molecule is provided by SEQ ID NO: 13. Additional exemplary MHC class II β1α1 polypeptides are disclosed in U.S. Pat. Nos. 6,270,772 and 8,377,447 and U.S. Pat. Application Publication Nos. 2008/0267987, and 2009/0280135; each of which is incorporated by reference in their entirety. The peptides can be replaced with one or more different antigens, such as those disclosed below.

B. Modified MHC Molecules

While the foregoing discussion uses as examples naturally occurring MHC class II molecules and the various domains of these molecules, one of skill in the art will appreciate that variants of these molecules and domains may be made and utilized in the same manner as described. Thus, reference herein to a domain of an MHC polypeptide or molecule (e.g., an MHC class II β1 domain or α1 domain) includes both naturally occurring forms of the referenced molecule, as well as molecules that are based on the amino acid sequence of the naturally occurring form, but which include one or more amino acid sequence variations. Such variant polypeptides may also be defined in the degree of amino acid sequence identity that they share with the naturally occurring molecule. Typically, MHC domain variants will share at least 80% sequence identity with the sequence of the naturally occurring MHC domain. More highly conserved variants will share at least 90% or at least 95% sequence identity with the naturally occurring sequence. Variants of MHC domain polypeptides also retain the biological activity of the naturally occurring polypeptide. For the purposes of this disclosure, that activity is conveniently assessed by incorporating the variant domain in the appropriate β1α1 polypeptide and determining the ability of the resulting polypeptide to inhibit antigen-specific T-cell proliferation in vitro.

Methods of determining antigen-specific T-cell proliferation are well known to one of skill in the art (see, e.g., Huan et al., J. Chem. Technol. Biotechnol. 80:2-12, 2005). In one example, T cells and APCs are incubated with stimulation medium only, Con A, or antigen with or without supplemental IL-2 (20 Units/ml) at 37° C. in 7% CO₂. The cultures are incubated for three days, the last 18 hours in the presence of [³H]thymidine. The cells are harvested and [³H]thymidine uptake assessed (for example by liquid scintillation counting).

Variant MHC domain polypeptides include proteins that differ in amino acid sequence from the naturally occurring MHC polypeptide sequence but which retain the specified biological activity. Such proteins may be produced by manipulating the nucleotide sequence of the molecule encoding the domain, for example by site-directed mutagenesis or the polymerase chain reaction. The simplest modifications involve the substitution of one or more amino acids for amino acids having similar biochemical properties. These so-called conservative substitutions are likely to have minimal impact on the activity of the resultant protein. Table 1 shows examples of amino acids which may be substituted for an original amino acid in a protein and which are regarded as conservative substitutions.

TABLE 1
Exemplary conservative amino acid substitutions

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

More substantial changes in biological function or other features may be obtained by selecting substitutions that are less conservative than those shown above, e.g., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions or deletions or additions may be assessed through the use of the described T-cell proliferation assay.

At the nucleic acid level, one of skill in the art will appreciate that the naturally occurring nucleic acid sequences that encode class II MHC domains may be employed in the expression vectors, but that the disclosure is not limited to such sequences. Any sequence that encodes a functional MHC domain may be employed, and the nucleic acid sequence may also be adapted to conform to the codon usage bias of the organism in which the sequence is to be expressed.

In some embodiments, the disclosed MHC molecules include modified MHC molecules that include one or more amino acid changes that decrease self-aggregation of native MHC polypeptides or β1α1 polypeptides. Modified MHC molecules of the disclosure are rationally designed and constructed to introduce one or more amino acid changes at a solvent-exposed target site located within, or defining, a self-binding interface found in the native MHC polypeptide. The self-binding interface that is altered in the modified MHC molecule typically includes one or more amino acid residues that mediate self-aggregation of a native MHC polypeptide, or of an “unmodified” β1α1 MHC molecule incorporating the native MHC polypeptide amino acid sequence. Although the self-binding interface is correlated with the primary structure of the native MHC polypeptide, this interface may only appear as an aggregation-promoting surface feature when the native polypeptide is isolated from the intact MHC complex and incorporated in the context of an “unmodified” β1α1 MHC molecule. In the case of exemplary MHC class II molecules described herein (e.g., comprising linked β1 and α1 domains), the native β1α1 structure only exhibits certain solvent-exposed, self-binding residues or motifs after removal of Ig-fold like β2 and α2 domains found in the intact MHC II complex. These same residues or motifs that mediate aggregation of unmodified β1α1 MHC molecules, are presumptively “buried” in a solvent-inaccessible conformation or otherwise “masked” (e.g., prevented from mediating self-association) in the native or progenitor MHC II complex (likely through association with the Ig-fold like β2 and α2 domains).

In some examples, an MHC molecule which has a reduced potential for aggregation in solution includes an “MHC component” in the form of a single chain polypeptide that includes multiple, covalently-linked MHC domain elements. These domain elements are typically selected from α1 and β1 domains of an MHC class II polypeptide, or portions thereof comprising an antigen (Ag)-binding groove/T-cell receptor (TCR) interface. The MHC component of the molecule is modified by one or more amino acid substitutions, additions, deletions, or rearrangements at a target site corresponding to a “self-binding interface” identified in a native MHC polypeptide component of an unmodified β1α1 MHC molecule. The modified β1α1 MHC molecule exhibits a markedly reduced propensity for aggregation in solution compared to aggregation exhibited by an unmodified, control β1α1 MHC molecule having the same fundamental MHC component structure, but incorporating the native MHC polypeptide defining the self-binding interface. Modified β1α1 MHC molecules with reduced potential for aggregation are described in detail in U.S. Pat. No. 8,377,447, incorporated by reference herein in its entirety.

The modified MHC molecules disclosed herein yield an increased percentage of monodisperse (monomeric) molecules in solution compared to a corresponding, unmodified MHC molecule (e.g., comprising the native MHC polypeptide and bearing the unmodified, self-binding interface). In certain embodiments, the percentage of unmodified MHC molecule present as a monodisperse species in aqueous solution may be as low as 1%, or more typically 5-10% or less of total MHC protein, with the balance of the unmodified MHC molecule being found in the form of higher-order aggregates. In contrast, modified MHC molecules disclosed herein yield at least 10%-20% monodisperse species in solution. In other embodiments, the percentage of monomeric species in solution will range from 25%-40%, often 50%-75%, up to 85%, 90%, 95%, or greater of the total MHC protein present, with a commensurate reduction in the percentage of aggregate MHC species compared to quantities observed for the corresponding, unmodified MHC molecules under comparable conditions.

MHC modification typically involves amino acid substitution or deletion at target sites for mutagenesis comprising a self-binding interface (including one or more amino acid residues, or a self-binding motif formed of several target residues). Within exemplary embodiments directed toward production of modified MHC molecule that include MHC class II β1α1 components, targeted residues for modification typically include hydrophobic residues or motifs, for example valine, leucine, isoleucine, alanine, phenylalanine, tyrosine, and tryptophan. These and other target residues may be substituted for any non-hydrophobic amino acid. Suitable amino acids for generating desired MHC molecule modifications include amino acids having aliphatic-hydroxyl side chains, such as serine and threonine; amino acids having amide-containing side chains, such as asparagine and glutamine; amino acids having aromatic side chains, such as phenylalanine, tyrosine, and tryptophan; and amino acids having basic side chains, such as lysine, arginine, and histidine.

In some examples, surface modification of an MHC molecule comprising an MHC class II component to yield much less aggregation-prone form can be achieved, for example, by replacement of one or more hydrophobic residues identified in the β-sheet platform of the MHC component with non-hydrophobic residues, for example polar or charged residues. In some examples, one or more hydrophobic amino acids of a central core portion of the β-sheet platform are modified, such as one or more of V97, 199, A101, F103, and L105 of a human MHC class II β1α1 construct (for example, SEQ ID NO: 13). In some examples, hydrophobic amino acids of a central core portion of the β-sheet platform include one or more amino acids at positions 6, 8, 10, 12, and 14 of a mature MHC class II α chain polypeptide or α1 domain (such as a human MHC class II DR α polypeptide). In one example the amino acids include one or more of V6, I8, A10, F12, and L14 of a mature human MHC class II α chain, such as a human DRB2 polypeptide. One of skill in the art can identify corresponding amino acids in other MHC class II molecules or β1α1 molecules. An exemplary modified MHC class II β1α1 RTL including substitution of aspartate residues for the hydrophobic amino acids corresponding to V97, I99, A101, F103, and L105 is DR2-5D (SEQ ID NO: 4).

In particular examples, one or more of the identified hydrophobic β-sheet platform amino acids is changed to either to a polar (for example, serine) or charged (for example, aspartic acid) residue. In some examples all five of V97, I99, A101, F103, and L105 (or corresponding amino acids in another MHC molecule) are changed to a polar or charged residue. In one example, each of V97, I99, A101, F103, and L105 are changed to an aspartic acid residue (e.g., SEQ ID NO: 4).

C. Expression and Purification of Recombinant MHC Molecules

In some embodiments, the MHC class II molecules disclosed herein are expressed in prokaryotic or eukaryotic cells from a nucleic acid construct. In their most basic form, nucleic acids encoding the MHC β1α1 polypeptides of the disclosure comprise first and second regions, having a structure B-A wherein A encodes the class II α1 domain and B encodes the class II β1 domain. Where a linker sequence is included, the nucleic acid may be represented as B-L1-A, wherein L1 is a nucleic acid sequence encoding the linker peptide. Where an antigenic peptide (P) is covalently linked to the MHC polypeptide, the nucleic acid molecule encoding this complex may be represented as P-B-A. A second linker sequence (L2) may be provided between the antigenic protein and the region B polypeptide (e.g., the linker sequence of SEQ ID NO: 5), such that the coding sequence is represented as P-L2-B-L1-A or P-L2-B-A. In all instances, the various nucleic acid sequences that comprise the RTL polypeptide (e.g., L1, L2, B, A and P) are operably linked such that the elements are situated in a single reading frame.

Nucleic acid constructs expressing these MHC polypeptides may also include regulatory elements such as promoters (Pr), enhancers, and 3′ regulatory regions, the selection of which will be determined based upon the type of cell in which the protein is to be expressed. When a promoter sequence is operably linked to the open reading frame, the sequence may be represented as Pr-B-A, or (if an antigen-coding region is included) Pr-P-B-A, wherein Pr represents the promoter sequence. The promoter sequence is operably linked to the P or B components of these sequences, and the B-A or P-B-A sequences (and any linkers) comprise a single open reading frame. The constructs are introduced into a vector suitable for expressing the MHC polypeptide in the selected cell type.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding construct. Suitable promoter sequences include the beta-lactamase, tryptophan (trp), phage T7 and lambda P_{L} promoters. Methods and plasmid vectors for producing heterologous proteins in bacteria or mammalian cells are described in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); and Ausubel et al., Short Protocols in Molecular Biology: A Compendium of Methods from Current Protocols in Molecular Biology, 4th ed., Wiley & Sons, 1999. In particular examples, the disclosed β1α1 polypeptides are expressed in a bacterial system, such as E. coli.

Expression of the MHC polypeptides in prokaryotic cells will result in polypeptides that are not glycosylated. Glycosylation of the polypeptides at naturally occurring glycosylation target sites may be achieved by expression of the polypeptides in suitable eukaryotic expression systems, such as mammalian cells.

Purification of the expressed protein is generally performed in a basic solution (typically around pH 10) containing 6M urea. Folding of the purified protein is then achieved by dialysis against a buffered solution at neutral pH (typically phosphate buffered saline at around pH 7.4 or Tris around pH 8.5).

D. Tumor Antigens

The disclosed MHC constructs include a tumor antigen, such as a prostate tumor antigen. Although the disclosure utilizes PSA antigens to exemplify the compositions, additional tumor antigens are also contemplated for use in the disclosed compositions and methods. Exemplary tumor antigens include, but are not limited to, those included in the Database of T-Cell Defined Human Tumor Antigens (available on the World Wide Web at http://cancerimmunity.org/peptide/).

In particular embodiments, the disclosed methods utilize MHC class II β1α1 molecules or MHC class II α1 molecules including a covalently linked tumor antigen (for example, a prostate tumor antigen, such as PSA or a portion thereof). As is well known in the art (see for example U.S. Pat. No. 5,468,481) the presentation of antigen in MHC complexes on the surface of APCs generally does not involve a whole antigenic peptide. Rather, a peptide located in the groove between the β1 and α1 domains (in the case of MHC II) is typically a small fragment of the whole antigenic peptide. As discussed in Janeway & Travers (Immunobiology: The Immune System in Health and Disease, 1997), peptides located in the peptide groove of MHC class II molecules typically at least 3-50 amino acids in length (such as 8-30, 10-25, or 15-23 amino acids in length). In some examples, the peptide located in the peptide groove of an MHC class II molecule is about 15-23 amino acids in length. Peptide fragments for loading into MHC molecules can be prepared by standard means, such as use of synthetic peptide synthesis machines.

In some examples, (e.g., treating prostate cancer), the disclosed antigens include a PSA peptide, such as a MHC class II restricted PSA peptide. In particular examples, the PSA peptide includes PSA amino acids 221-240 (e.g., SEQ ID NO: 1), PSA amino acids 221-236 (e.g., amino acids 1-16 of SEQ ID NO: 1), or PSA amino acids 171-190 (e.g., SEQ ID NO: 3). Variants of the disclosed PSA peptides can also be used in the compositions and methods disclosed herein. In some examples, the variant PSA peptide includes one or more (such as 1, 2, 3, 4, 5, 6, 7, 8, 9, 10, or more) substitutions, additions, deletions, and/or insertions compared to the wild type or native peptide. An exemplary PSA peptide variant is PSA₂₂₁₋₂₄₀ 67S (SEQ ID NO: 2), which has a substitution of serine for the cysteine at amino acid position 234 of the full-length PSA protein. Without being bound by theory, it is believed that substitution of the cysteine for another amino acid (such as serine), may improve recombinant expression of constructs including this peptide compared to the native PSA₂₂₁₋₂₄₀ sequence. Other PSA peptides include PSA₁₆₉₋₁₈₁ (KKLQCVDLHVISN; SEQ ID NO: 14), PSA₂₂₁₋₂₃₃ (GVLQGITSWGSEP; SEQ ID NO: 15), PSA₁₇₁₋₁₇₉ (LQCVDLHVI; SEQ ID NO: 16), and PSA₂₂₃₋₂₃₁ (LQGITSWGS; SEQ ID NO: 17). Additional exemplary PSA MHC class II restricted peptides and variants of use in the disclosed compositions and methods are described in U.S. Pat. No. 8,435,507, which is incorporated by reference herein.

In some examples (such as MHC β1α1 constructs), the antigen is covalently linked to the MHC class II molecule by operably linking a nucleic acid sequence encoding the selected antigen to the 5′ end of the construct encoding the MHC protein such that, in the expressed peptide, the antigenic peptide domain is linked to the amino-terminus of the β1 domain. In other examples, (such as MHC α1 constructs), the selected antigen is operably linked to the 3′ end of a construct encoding the MHC α1 domain. One convenient way of obtaining this result is to incorporate a sequence encoding the antigen into the PCR primers used to amplify the MHC coding regions. Typically, a nucleic acid encoding a linker peptide sequence will be included between the nucleic acids encoding the antigenic peptide and the MHC polypeptide. As discussed above, the purpose of such linker peptides is to provide flexibility and permit proper conformational folding of the peptides. For linking antigens to the MHC polypeptide, the linker should be sufficiently long to permit the antigen to fit into the peptide groove of the MHC polypeptide. Again, this linker may be conveniently incorporated into the PCR primers. However, it is not necessary that the antigenic peptide be ligated exactly at the 5′ end of the MHC β1α1 coding region. For example, the antigenic coding region may be inserted within the first few (typically within the first 10) codons of the 5′ end of the MHC β1α1 coding sequence.

In other examples, the β1α1 molecules are expressed and purified in an empty form (e.g., without attached antigenic peptide), and the antigen is loaded into the molecules using standard methods. Methods for loading antigenic peptides into MHC molecules are described in, for example, U.S. Pat. No. 5,468,481, herein incorporated by reference. Such methods include simple co-incubation of the purified MHC molecule with a purified preparation of the antigen.

In some examples, the antigen is covalently linked to the MHC molecule by a disulfide bond. In some examples, the disulfide linkage is formed utilizing a naturally occurring cysteine residue in the MHC polypeptide (such as a cysteine residue in the MHC class II β1 domain). In some examples, the cysteine residue is in the MHC class II β1 domain. In particular examples, the disulfide linkage utilizes Cys 11 and/or Cys 75 of a MHC β1α1 polypeptide (for example, SEQ ID NO: 4). One of skill in the art can identify corresponding cysteine residues in other MHC β1α1 MHC polypeptides. In other examples, the disulfide linkage is formed utilizing a non-naturally occurring cysteine residue in the MHC polypeptide, such as a cysteine residue introduced in the MHC polypeptide by mutagenesis. In further examples, the disulfide linkage is formed utilizing a naturally occurring cysteine residue in the peptide antigen. In still further examples, the disulfide linkage is formed utilizing a non-naturally occurring cysteine residue in the peptide antigen, such as a cysteine residue introduced in the peptide antigen by mutagenesis. Exemplary MHC molecules wherein the antigen is covalently linked by a disulfide bond are described in U.S. Pat. App. Publ. No. 2013/0171179, incorporated herein by reference in its entirety.

In one non-limiting example, empty β1α1 molecules may be loaded by incubation with an excess (e.g., a 2-fold, 5-fold, 10-fold, or more molar excess) of peptide at room temperature, for 24 hours or more. Thereafter, excess unbound peptide may be removed by dialysis (for example, dialysis against PBS at 4° C. for 24 hours). Peptide binding to β1α1 can be detected and/or quantified by silica gel thin layer chromatography (TLC) using radiolabeled peptide or by gel electrophoresis. Based on such quantification, the loading may be altered (e.g., by changing the molar excess of peptide or the time of incubation) to obtain the desired result.

### III. Methods of Treating or Inhibiting Cancer

Disclosed herein are methods for treating or inhibiting cancer (such as prostate cancer) in a subject. The methods include administering one or more of the disclosed MHC class II constructs covalently linked to a tumor antigen (such as a prostate tumor antigen) to a subject. Exemplary MHC class II constructs (such as β1α1 RTLs) and tumor antigens (such as prostate specific antigens) are described in detail in Section II, above.

Exemplary RTLs that can be administered to a subject to treat or inhibit prostate cancer include, but are not limited to, those disclosed in SEQ ID NOs: 6-10. In some embodiments, methods of treating or inhibiting prostate cancer in a subject include selecting a subject with prostate cancer and administering to the subject an effective amount of a disclosed RTL. In some examples, the subject is a mammalian subject (such as a human subject, a primate subject, or a rodent subject).

In some embodiments, the disclosed methods further include measuring or assessing response of the cancer to the treatment, such as an increase in survival (such as overall survival, progression-free survival, or metastasis-free survival) or a decrease in the size, volume, or number of tumors.

In some examples, treating or inhibiting prostate cancer in a subject includes an increase in survival (such as at least about a 20% increase, at least about a 50% increase, at least about a 75% increase, at least about an 80% increase, at least about a 90% increase, at least about a 1.5-fold increase, at least about a 2-fold increase, at least about a 3-fold increase, or at least about a 5-fold increase) in the subject as compared to a control. In other examples, treating or inhibiting prostate cancer in a subject includes a decrease (such as at least about a 20% decrease, at least about a 50% decrease, at least about a 75% decrease, at least about an 80% decrease, or at least about a 90% decrease) in one or more measures of tumor size (e.g., tumor base area or tumor volume) or the number of tumors in a subject as compared to a control.

The control can be any suitable control against which to compare the subject. In some embodiments, the control is a reference value or ranges of values. For example, the reference value can be derived from the average values obtained from a group of normal control subjects (for example, subjects without prostate cancer). In further examples, the reference value is derived from the average values obtained from a group of subjects with prostate cancer, for example, an untreated subject or a subject treated with vehicle alone. In other examples, the control is obtained from the same subject, for example, a subject with prostate disease prior to treatment.

The disclosed RTLs for use in treating or inhibiting prostate cancer can be formulated as a pharmaceutical composition. Pharmaceutical compositions that include one or more of the DR2/PSA RTLs disclosed herein (such as one or more of SEQ ID NOs: 6-10) can be formulated with an appropriate solid or liquid carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21′ Edition (2005). For instance, parenteral formulations usually include injectable fluids that are pharmaceutically and physiologically acceptable fluid vehicles such as water, physiological saline, other balanced salt solutions, aqueous dextrose, glycerol, or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

The dosage form of the pharmaceutical composition will be determined by the mode of administration chosen. For instance, in addition to injectable fluids, topical, inhalation, oral and suppository formulations can be employed. Topical preparations can include ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

In some examples, the pharmaceutical composition may be administered by any means that achieve their intended purpose. Amounts and regimens for the administration of the selected DR2/PSA RTL(s) will be determined by the attending clinician. Effective doses for therapeutic application will vary depending on the nature and severity of the condition to be treated, the particular RTL selected, the age and condition of the patient, and other clinical factors. Typically, the dose range will be from about 0.1 μg/kg body weight to about 100 mg/kg body weight. Other suitable ranges include doses of from about 100 μg/kg to 10 mg/kg body weight or about 500 μg/kg to about 5 mg/kg. The dosing schedule may vary from once a week to daily depending on a number of clinical factors, such as the subject's sensitivity to the protein. Examples of dosing schedules are about 1 mg/kg administered once a week, twice a week, three times a week or daily; a dose of about 5 mg/kg once a week, twice a week, three times a week or daily; or a dose of about 10 mg/kg once a week, twice a week, three times a week or daily.

The pharmaceutical compositions that include a one or more of the disclosed RTL molecules can be formulated in unit dosage form, suitable for individual administration of precise dosages. In one specific, non-limiting example, a unit dosage can contain from about 1 ng to about 500 mg of DR2/PSA RTL (such as about 10 ng to 50 mg or about 1 mg to 100 mg, for example, about 25 mg, about 60 mg, about 75 mg, or about 100 mg). The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The compounds and pharmaceutical compositions of this disclosure can be administered to humans or other animals on whose tissues they are effective in various manners such as topically, orally, intravenously, intramuscularly, intraperitoneally, intranasally, intradermally, intrathecally, subcutaneously, via inhalation, or via suppository. In one example, the compounds are administered to the subject subcutaneously. In another example, the compounds are administered to the subject intravenously. The particular mode of administration and the dosage regimen will be selected by the attending clinician, taking into account the particulars of the case (e.g., the subject, the disease, the disease state involved, and other factors). Treatment can involve monthly, bi-monthly, weekly, daily or multi-daily doses of compound(s) over a period of a few days to months, or even years.

The present disclosure also includes combinations of one or more of the disclosed RTLs in combination with one or more other agents useful in the treatment of prostate cancer. For example, the compounds of this disclosure can be administered in combination with effective doses of one or more therapies for prostate cancer, including but not limited to, surgery, chemotherapeutic drug treatment, radiation, gene therapy, hormone therapy (such as androgen depletion therapy), immunotherapy, and antisense oligonucleotide therapy. Examples of useful chemotherapeutic drugs include, but are not limited to, microtubule binding agents, DNA intercalators or cross-linkers, DNA synthesis inhibitors, DNA and/or RNA transcription inhibitors, enzyme inhibitors, gene regulators, enzymes, antibodies, angiogenesis inhibitors, or combinations of two or more thereof. Examples of prostate cancer therapies include abiraterone acetate (e.g., Zytiga®), bicalutamide (e.g., Casodex®), cabazitaxel (e.g., Jevtana®), degarelix, docetaxel (e.g., Taxotere®), enzalutamide (e.g., Xtandi®), flutamide, goserelin acetate (e.g., Zoladex®), leuprolide acetate (e.g., Lupron®, Lupron Depot®, Viadur®), mitoxantrone hydrochloride, prednisone, sipuleucel-T (e.g., Provenge®), and radium 223 dichloride (e.g., Xofigo®). The term “administration in combination” or “co-administration”refers to both concurrent and sequential administration of the active agents or therapies.

The following examples are provided to illustrate certain particular features and/or embodiments. These examples should not be construed to limit the disclosure to the particular features or embodiments described.

### Example 1

### T Cell Response to Prostate Specific Antigen

The T cell response was studied in a mouse model utilizing Transgenic Adenocarcinoma of Mouse Prostate (TRAMP) tumors expressing the human tumor antigen PSA (TRAMP-PSA) in transgenic Human Leukocyte Antigen (HLA)-DR2b mice (Klyushnekova et al., J. Immunol. 182:1242-1246, 2009). TRAMP-PSA DR2b tg×C57BL/6-PSA F1 mice (hereafter termed F1 mice) express the full MHC haplotype of B6 mice in addition to the DR2b transgene. This allows the TRAMP-PSA tumor, which is of B6 origin, to grow and allows observation of the effect of DR2b on tumor growth. Two peptides from PSA, PSA₁₇₁₋₁₉₀ and PSA₂₂₁₋₂₄₀, can be presented by DR2b but no peptides from PSA are presented by I-A^{b} (Klyushnenkova et al., Clin. Cancer Res. 11:2853-2861, 2005).

It was originally hypothesized that PSA presentation by both class I and class II in the F1 mice would result in strong tumor rejection. Unexpectedly, animals expressing DR2b had progressively growing tumors expressing the PSA tumor antigen and no CD8 T cell response to PSA. Animals lacking the DR2b transgene rejected their tumors. The small amount of palpable tumor tissue at the site of injection lacked expression of PSA and these animals had a strong CD8 T cell response to PSA (Klyushnenkova et al., Clin. Cancer Res. 11:2853-2861, 2005).

Since DR2b can present PSA peptides and I-A^{b} cannot, the data strongly suggested that the suppression of the CD8 T cell response to PSA that would otherwise reject the tumor was due to a process that involves CD4 T cells that are recognizing PSA in the context of DR2b. One candidate for this suppressive process is the T_{reg}population. Further, the antigen specificity of the process suggested there are PSA specific T_{reg} in the DR2b animals (Klyushnenkova et al., Clin. Cancer Res. 11:2853-2861, 2005).

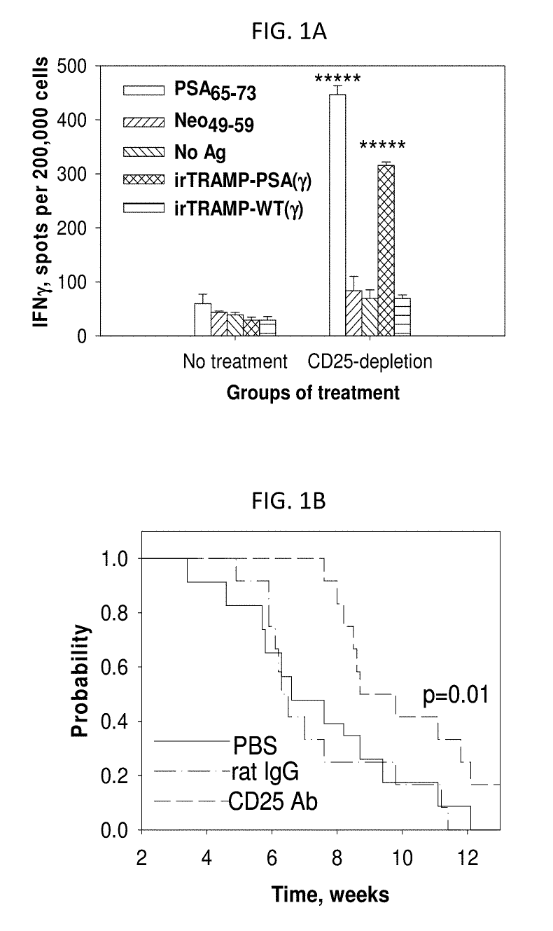

In one experiment, CD25⁺ cells were depleted prior to tumor inoculation in F1 mice. As shown in FIGS. 1A and 1B, this depletion removed the suppressive phenotype (FIG. 1A), restored PSA specific CD8 T cells to levels found in B6 mice and resulted in tumor rejection (FIG. 1B). T_{reg} depletion before tumor inoculation thus reversed the suppressive phenotype.

Depleting CD25 cells removes this subpopulation (along with all Treg) and restores tumor rejection. This hypothesis is supported by a recent study showing in TRAMP mice that Treg for a self-tissue antigen are selected in the thymus, populate the periphery and accumulate in tumors of the same tissue origin (Klyushnenkova et al., Clin. Cancer Res. 11:2853-2861, 2005).

### Example 2

### Recombinant T Cell Ligand-Prostate Specific Antigen Peptide

An RTL comprising DR2b α1 and β1 chains covalently attached to a PSA peptide was generated. The structure-based design of the RTL is further described in detail in previous publications (Huan et al., J. Immunol. 172:4556-4566, 2004; Burrows et al., Prot. Eng. 12:771-779, 1999; Burrows et al., J. Immunol. 161:5987-5996, 1998; Huan et al., J. Chem. Technol. Biotechnol. 80:2-12, 2005; and Chang et al., J. Biol. Chem. 276:24170-2417, 2001) and described above. RTLs are believed to block CD4 T cell actions in vitro and in vivo by cognate interaction with TCR, but other mechanisms for their CD4 blocking activity have recently been described (Vandenbark et al., J. Autoimmunity 40:96-110, 2013).

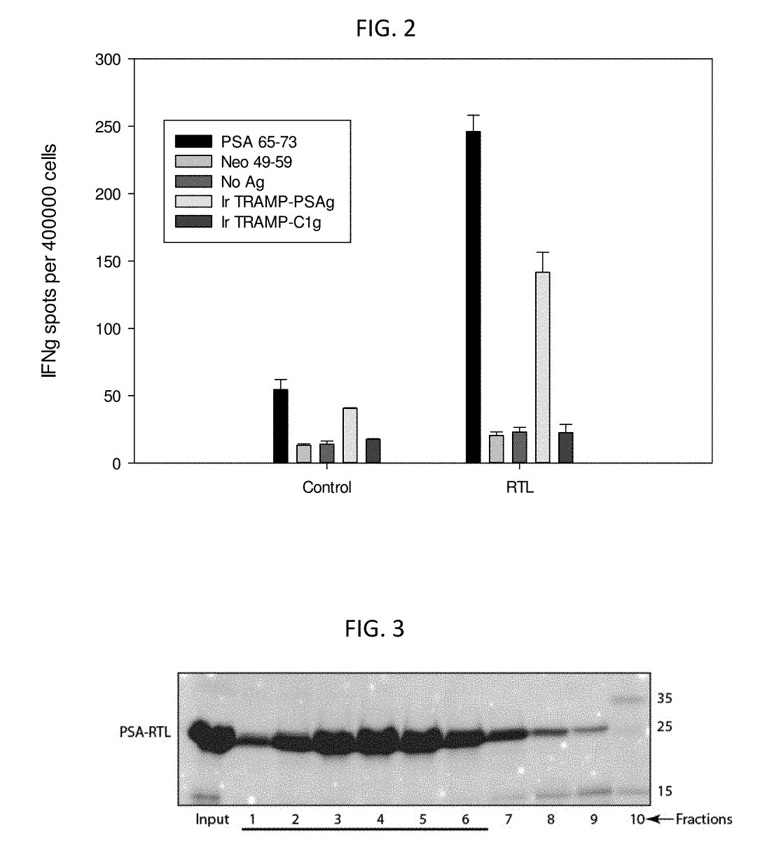

Blocking CD4 suppressor T cells that are specific for PSA using an RTL could increase PSA-specific CTL and restore tumor rejection in F1 mice using the model described in Klyushnenkova et al. (J. Immunol. 182:1242-1246, 2009). A DR2b-PSA₂₂₁₋₂₃₆ RTL was generated. The particular RTL used was created by loading PSA₂₂₁₋₂₃₆ into empty α1 and β1 RTL constructs rather than by a construct comprising covalently attached peptide. As shown in FIG. 2, the administration of RTL/PSA₂₂₁₋₂₃₆ to DR2b mice bearing TRAMP-PSA resulted in restoration of the CD8 T cell response to PSA and irradiated TRAMP-PSA. These data are supportive of the hypothesis that PSA-specific T_{reg} contribute to the suppression of the CTL response to TRAMP-PSA and provide evidence that RTL restore the CTL response to PSA.

### Example 3

### Expression and Purification of DR2/PSA RTL

DR2/PSA RTL peptide construction, cloning and expression. DNA coding for the single chain DR2/PSA₂₂₁₋₂₄₀ RTL protein was cloned in the pET cloning system (Novagen) by using the NcoI and XhoI restriction sites, expressed in Escherichia coli, and purified using standard chromatographic methods. The expressed protein includes a β1 domain from DRB1*1501 or DR2-5D at the N-terminus linked to the C-terminus of the α1 domain of the DRA through a flexible linker. Antigenic peptide of SEQ ID NO: 1 or SEQ ID NO: 2 were inserted up-stream of the β1 domain using two oligonucleotide primers as described previously. The loading efficiency of peptide using this method approached 100%.

DR2/PSA RTL protein purification and characterization. E. coli harboring the PSA-RTL plasmid were inoculated into 4 1 L flasks of LB medium supplemented with 50 μg/ml of Carbenicillin. Cultures were incubated until they reached an OD₆₀₀ between 0.6 and 0.8. At this point, 2 mM IPTG was added to the cultures and incubated an additional 4 hours. Cells were harvested at 7000 rpm at 4° C. and the pellets frozen until use. Bacterial pellets were then resuspended in sonication buffer and sonicated to release inclusion bodies. Once enriched, inclusion bodies were solubilized in a buffer containing 6 M urea overnight at 4° C. Lysate was centrifuged at 40,000×g and supernatant was filtered and applied to an anion exchange column. Material was eluted stepwise with increasing concentrations of NaCl and peaks containing the PSA-RTL were collected and analyzed by electrophoresis. Fractions were pooled together and concentrated to an OD₂₈₀ of 5 and 2 ml samples were applied to a SUPERDEX 75 16/60 size exclusion column previously equilibrated with a buffer containing 6 M urea and the same concentration of NaCl at which the protein was eluted from the anion exchange column. Fractions were collected and analyzed by OD₂₈₀ and by electrophoresis (FIG. 3). Fractions containing the PSA-RTL protein were identified and pooled. For refolding, the protein concentration was adjusted to 0.2 mg/ml and dialyzed against 20 mM Tris, pH 8.5 until the theoretical urea concentration was in the picomolar concentration range. Fractions 1 through 6 (FIG. 3) were pooled and subjected to dialysis and refolding. After purification and refolding, the protein was aliquoted, flash-frozen and stored at −80° C.

### Example 4

### Determining Efficacy of DR2/PSA RTL in a Mouse Model of Prostate Cancer

This example describes methods that can be used to test DR2/PSA RTLs for treatment of prostate cancer in a mouse model. However, one skilled in the art will appreciate that methods that deviate from these specific methods can also be used to assess the efficacy of the RTL constructs in mice.

F1 male mice (8-12 weeks old) can be inoculated s.c. with 3×10⁶ TRAMP-PSA tumor cells. RTL/PSA₂₂₁₋₂₄₀, RTL/PSA₁₇₁₋₁₉₀, irrelevant control RTL/MOG₃₅₋₅₅ or vehicle can be injected s.c. daily during 5 days preceding tumor inoculation and daily on days 9-11 after tumor inoculation (100 μg/dose in 100 μl vehicle). Mice are euthanized two weeks after tumor inoculation.

Spleens and DLN can be harvested for the evaluation of the CTL response to PSA by several different assays. The total number of PSA-specific CTL can be determined using a PSA₆₅₋₆₃ dextramer reagent that has previously been used to enumerate the number of CTL recognizing PSA. The functional capability of PSA-specific CTL can be examined using ELISPOT and intracellular cytokine staining (ICS). The ELISPOT assay for IFNγ can be performed as described in Klyushnenkova et al. (J. Immunol. 182:1242-1246, 2009). Intracellular cytokine staining can be performed using splenocytes and DLN cells. Cells can be stimulated in vitro with 10 μg/ml PSA₆₅₋₇₃. Unstimulated cultures and PMA/ionomycin stimulation can serve as negative and positive controls respectively. ICS staining after treatment with Brefeldin and permeabilization can be performed using appropriate paired antibodies and subtype controls for IFNγ and TNFα. These experiments should result in a strong specific CTL response to PSA in response to RTL administration. Comparisons between groups can be performed by Analysis of Variance (ANOVA).

In addition, repeated administration of the PSA-RTL can be used. F1 male mice (8-12 weeks old) can be inoculated with 3×10⁶ TRAMP-PSA tumor cells s.c. RTL treatment can be performed initially as described above prior to and immediately after inoculation with tumor cells. Further, animals can receive a weekly dose of 100 μg of RTL/PSA₂₂₁₋₂₄₀ or RTL/PSA₁₇₁₋₁₉₀ s.c. throughout the period of tumor monitoring, typically up to 20 weeks. Tumor growth can be monitored. Tumor base area can be calculated by measuring the two greatest bisecting diameters of the tumor and multiplying these values. A tumor base area of 100 mm² can be used as a surrogate end point for survival. All tumor measurements can be performed by an investigator blinded to the treatment group. Survival analysis can be performed using MedCalc software; differences between groups can be analyzed by log rank test.

Other experiments can be used to determine if RTL administration can impact mice with established TRAMP-PSA tumor. These experiments can be performed as above except that RTL administration can begin on day 3 after TRAMP-PSA inoculation. Animals on day 3 can receive 5 doses of 100 μg of RTL/PSA₂₂₁₋₂₄₀, RTL/PSA₁₇₁₋₁₉₀, irrelevant control RTL/MOG₃₅₋₅₅ or vehicle s.c. They can then receive weekly doses of RTL/PSA₂₂₁₋₂₄₀ or RTL/PSA₁₇₁₋₁₉₀. Tumor area can be determined and analyzed as above.

### Example 5

### Treatment of Prostate Cancer

This example describes exemplary methods for treating or inhibiting prostate cancer in a subject. However, one of skill in the art will appreciate that methods that deviate from these specific methods can also be used to treat prostate cancer in a subject.

Subjects having prostate cancer are selected. Subjects are treated weekly (for example, by intravenous administration) with an MHC class II β1α1 polypeptide covalently linked to a PSA peptide antigen (for example, PSA₂₂₁₋₂₄₀ or PSA₁₇₁₋₁₉₀) or other RTLs as disclosed herein, at doses of 0.1 mg/kg to 25 mg/kg. Subjects are assessed for signs or symptoms of prostate cancer, prior to initiation of therapy, periodically during the period of therapy, and/or at the end of the course of treatment.

The effectiveness of the therapy to treat or inhibit prostate cancer in a subject can be demonstrated by an improvement in one or more signs or symptoms of prostate cancer (such as size, volume, and/or number of tumors and/or survival), for example, compared to one or more untreated subjects with prostate cancer, one or more subjects with prostate cancer prior to treatment (for example, the same subject prior to treatment), or one or more subjects with prostate cancer treated with placebo (e.g., vehicle only).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A composition comprising:
a major histocompatibility complex (MHC) class II molecule comprising covalently linked first and second domains, wherein the first domain is an MHC class II β1 domain and the second domain is an MHC Class II α1 domain, wherein the amino terminus of the α1 domain is covalently linked to the carboxy terminus of the β1 domain, wherein the MHC molecule does not comprise an MHC class II α2 domain or an MHC Class II β2 domain; and
an antigenic determinant comprising at least a portion of a prostate specific antigen covalently linked to the first domain.

2. The composition of claim 1, wherein the covalent linkage between the first domain and the second domain comprises a polypeptide linker.

3. The composition of claim 1, wherein the antigenic determinant is covalently linked to the first domain by a polypeptide linker or a disulfide bond.

4. The composition of claim 1, wherein the MHC molecule comprises a human MHC molecule.

5. The composition of claim 1, wherein the MHC molecule comprises an HLA-DR MHC molecule.

6. The composition of claim 1, wherein the MHC molecule is modified by substitution of one or more hydrophobic amino acids within a β-sheet platform of the MHC molecule, such that the MHC molecule exhibits reduced aggregation in solution compared to aggregation exhibited by an unmodified MHC molecule with a wild-type β-sheet platform.

7. The composition of claim 6, wherein the one or more hydrophobic amino acids are selected from V6, I8, A10, F12, L14, and a combination of two or more thereof of the MHC class II α1 domain, and wherein the one or more hydrophobic amino acids are substituted with a non-hydrophobic amino acid.

8. The composition of claim 7, wherein all of V6, I8, A10, F12, and L14 are substituted with a non-hydrophobic amino acid.

9. The composition of claim 7, wherein the non-hydrophobic amino acid is a polar or a charged amino acid.

10. The composition of claim 9, wherein the non-hydrophobic amino acid is serine or aspartic acid.

11. The composition of claim 1, wherein the antigenic determinant comprises or consists of the amino acid sequence of any one of SEQ ID NOs: 1 to 3.

12. The composition of claim 1, wherein the composition comprises a polypeptide comprising the amino acid sequence of any one of SEQ ID NOs: 6, 8, or 10.

13. The composition of claim 1, wherein the composition comprises a nucleic acid molecule comprising the nucleotide sequence of SEQ ID NO: 7 or SEQ ID NO: 9.

14. The composition of claim 13, wherein the nucleic acid sequence is operably linked to a promoter.

15. The composition of claim 1, further comprising a pharmaceutically acceptable carrier.

16. A method for treating or inhibiting prostate cancer in a subject, comprising administering an effective amount of the composition of claim 1 to the subject.

17. The method of claim 16, further comprising selecting the subject with prostate cancer for treatment.

18. The method of claim 16, further comprising measuring response of the prostate cancer to treatment.

19. The method of claim 16, further comprising administering to the subject a second therapy for the prostate cancer that is not an MHC molecule.


SEQUENCE LISTING (17 sequences)

SEQ ID NO: 1 - protein, 20 aa, Artificial Sequence
  Synthetic polypeptide
GVLQGITSWGSEPCALPERP

SEQ ID NO: 2 - protein, 20 aa, Artificial Sequence
  Synthetic polypeptide
GVLQGITSWGSEPSALPERP

SEQ ID NO: 3 - protein, 20 aa, Artificial Sequence
  Synthetic polypeptide
LQCVDLHVISNDVCAQVHPQ

SEQ ID NO: 4 - protein, 175 aa, Artificial Sequence
  Synthetic polypeptide
PRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITN

SEQ ID NO: 5 - protein, 15 aa, Artificial Sequence
  Synthetic polypeptide linker
GGGGSLVPRGSGGGG

SEQ ID NO: 6 - protein, 211 aa, Artificial Sequence
  Synthetic polypeptide
MGVLQGITSWGSEPCALPERPGGGGSLVPRGSGGGGPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITN

SEQ ID NO: 7 - DNA, 644 bp, Artificial Sequence
  Synthetic nucleic acid construct
ccatgggtgtgcttcaaggtatcacgtcatggggcagtgaaccatgtgccctgcccgaaaggcctggaggtggaggctcactagtgccccgaggctctggaggtggaggcccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaattaactcgag

SEQ ID NO: 8 - protein, 211 aa, Artificial Sequence
  Synthetic polypeptide
MGVLQGITSWGSEPSALPERPGGGGSLVPRGSGGGGPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITN

SEQ ID NO: 9 - DNA, 644 bp, Artificial Sequence
  Synthetic nucleic acid construct
ccatgggtgtgcttcaaggtatcacgtcatggggcagtgaaccatcagccctgcccgaaaggcctggaggtggaggctcactagtgccccgaggctctggaggtggaggcccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaattaactcgag

SEQ ID NO: 10 - protein, 210 aa, Artificial Sequence
  Synthetic polypeptide
LQCVDLHVISNDVCAQVHPQGGGGSLVPRGSGGGGPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITN

SEQ ID NO: 11 - protein, 9 aa, Artificial Sequence
  Synthetic polypeptide
HCIRNKSVI

SEQ ID NO: 12 - protein, 11 aa, Artificial Sequence
  Synthetic polypeptide
SSPVNSLRNVV

SEQ ID NO: 13 - protein, 175 aa, Artificial Sequence
  Synthetic polypeptide
PRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 14 - protein, 13 aa, Artificial Sequence
  Synthetic polypeptide
KKLQCVDLHVISN

SEQ ID NO: 15 - protein, 13 aa, Artificial Sequence
  Synthetic polypeptide
GVLQGITSWGSEP

SEQ ID NO: 16 - protein, 9 aa, Artificial Sequence
  Synthetic polypeptide
LQCVDLHVI

SEQ ID NO: 17 - protein, 9 aa, Artificial Sequence
  Synthetic polypeptide
LQGITSWGS